(19) United States
(12) Patent Application Publication
(10) Pub. No.: US 2018/0177436 A1
(43) Pub. Date: Jun. 28, 2018

(54) SYSTEM AND METHOD FOR REMOTE MONITORING FOR ELDERLY FALL PREDICTION, DETECTION, AND PREVENTION

(71) Applicant: Lumo BodyTech, Inc, Mountain View, CA (US)

(72) Inventors: Andrew Robert Chang, Sunnyvale, CA (US); Chung-Che Charles Wang, Palo Alto, CA (US)

(21) Appl. No.: 15/850,147

(22) Filed: Dec. 21, 2017

Related U.S. Application Data
(60) Provisional application No. 62/438,149, filed on Dec. 22, 2016.
(60) Provisional application No. 62/522,017, filed on Jun. 19, 2017.

(51) Int. Cl. A61B 5/11; A61B 5/00

(52) U.S. Cl. CPC A61B 5/1117; A61B 5/112; A61B 5/02055; A61B 5/746; A61B 5/7465

(57) ABSTRACT

A system and method for remote monitoring for elderly fall prediction, detection, and prevention that includes collecting sensor data at a biomechanical sensing device coupled to a user; performing biomechanical analysis on the sensor data and thereby generating mobility metrics of the user, wherein performing biomechanical analysis including quantifying a set of gait dynamics as a component of the mobility metrics and generating a user activity graph as a component of the mobility metrics; processing the mobility metrics in a risk assessment model and thereby generating a fall risk assessment; and detecting a trigger condition and triggering a response to the fall risk assessment.

### CROSS-REFERENCE TO RELATED APPLICATIONS

This Application claims the benefit of U.S. Provisional Application No. 62/438,149 filed on 22 Dec. 2016, and U.S. Patent Application No. 62/522,017, filed on 19 Jun. 2017, both of which are incorporated in their entireties by this reference.

### TECHNICAL FIELD

This invention relates generally to the field of biomechanical monitoring, and more specifically to a new and useful system and method for remote monitoring for elderly fall prediction, detection, and prevention.

### BACKGROUND

Falls are a major problem among elderly and for individuals in poor health. Despite many efforts in elderly care institutions and in hospitals, falls still pose a serious health risk. Complicating the prevention of falls, current trends show that most senior citizens prefer to live at home, and often times they are alone and more at-risk to injury. Even within a senior care nursing home, nurses cannot monitor an elderly person 24 hours a day, and attention must be split among numerous residents.

While there are products that allow a user to press a button when they've fallen and cannot get up, there are limited tools to inform an individual if and when they are at risk of falling.

Additionally, the current methods of obtaining the information on human movement has primarily been done by a patient coming into the clinic for examination, evaluations and observations done by a nurse at an elderly care residence, or in-home nurse visits if an elderly person was living at home. Currently, the quantification of human movement is mostly a hands-on manual observation process. This generalized assessment performed over long durations means that doctors and caretakers have few ways to reasonably help an individual other than to recommend general tips. In some cases, guidance to avoid walking may prompt an individual to avoid moving, which has its own negative impact on health and wellbeing.

Thus, there is a need in the biomechanical monitoring field to create a new and useful system and method for remote monitoring for elderly fall prediction, detection, and prevention. This invention provides such a new and useful system and method.

### BRIEF DESCRIPTION OF THE FIGURES

FIG. 1 is a schematic representation of a system of a preferred embodiment;

FIG. 2 is a schematic representation of a variation of a risk analysis model being used in generating a response;

FIG. 3 is a flowchart representation of a method of a preferred embodiment;

FIG. 4 is an exemplary chart representation of asymmetric step detection;

FIG. 5 is an exemplary chart representation of quantifying a double-stance ratio;

FIG. 6 is an exemplary chart comparison representing shuffle detection;

FIG. 7 is a schematic representation of a variation of a risk analysis model being used in recommending a predicted rest time; and

FIG. 8 is a schematic representation of providing a rehabilitation progress report.

### DESCRIPTION OF THE EMBODIMENTS

The following description of the embodiments of the invention is not intended to limit the invention to these embodiments but rather to enable a person skilled in the art to make and use this invention.

### 1. Overview

A system and method for remote monitoring for elderly fall prediction, detection, and prevention functions to apply a biomechanical sensing platform to interpreting movement characteristics to understanding an individuals risk of falling, mobility history, and/or recovery or changes in mobility. In a preferred embodiment, the system and method can be used to predict risk of falling and even predict or identity aspects that can reduce the risk of falling. In another embodiment variation, the system and method can track on mobility history, which may be used for detecting falls, stumbles, moments of imbalance, or other suitable mobility-related events and reporting those to appropriate people and/or systems. As another embodiment variation, the system and method can be used to promote recovery, possibly tracking mobility progress and assisting or even directing a patient for mobility improvement.

The system and method are preferably implemented in connection with a biomechanical sensing platform. The biomechanical sensing platform may provide a tool for nurses and family members to remotely monitor the movements of their elder patients in a non-intrusive but helpful, reliable, and scalable manner. In one variation, the platform can analyze the mobility quality of each patient throughout the entire day, providing a detailed breakdown of the specific biomechanical issues a patient is experiencing, provide real-time feedback to the patient, or send an alert if medical attention is required.

The biomechanical sensing platform can automatically and objectively quantify the mobility metrics of a user. In particular, the mobility metrics generated through the system and method can include gait dynamics (e.g., walking gait dynamics, running gait dynamics, etc.) and an overall activity graph. An activity graph is preferably a data representation that characterizes different time-based activity states detected for a user over a time period (e.g., during the course of a day). Activity states can include classifying activities and events like lying down, sitting down, standing, walking, running, using a walker, using a wheel chair, going up stairs, going down stairs, stumbling, falling, being unbalanced, and the like. Mobility metrics that convey mobility quality can help researchers, family members, and the medical and senior care communities provide better care to aging populations. This data can be used to identify which seniors are at high risk of falling or have fallen down and suffered an injury.

The system and method can be used to address various healthcare issues such as: identifying senior citizens who are at a high risk of falling down or suffering from other health risks; preventing high risk elderly people from falling; detecting falls; providing real-time feedback on gait re-training; performing remote monitoring; monitoring workers compensation claims; and/or other suitable applications.

As one potential benefit, the system and method can preferably proactively alert or warn a user or caretaker of falling risk prior to the occurrence of a fall.

As another potential benefit, the system and method can generate a deep understanding of mobility quality and changes in mobility quality such that the system and method can be used to drive feedback customized to the situation. The system and method could have multiple interactions such as user feedback, family member communication, caretaker communication, automatic emergency contact, mobility coaching, rest prediction, and/or other features that can be customized to each user's current situation.

As another potential benefit, the system and method can preferably be used without depending on large volumes of training data. By being based on detectable and quantifiable biomechanical aspects, the system and method can be readily helpful to users. Related to this, the initial usefulness of the system and method additionally enables data driven techniques to be developed and integrated in parallel that use the collected data of the system and method. The data driven analysis may enable alternative risk analysis approaches.

### 2. System

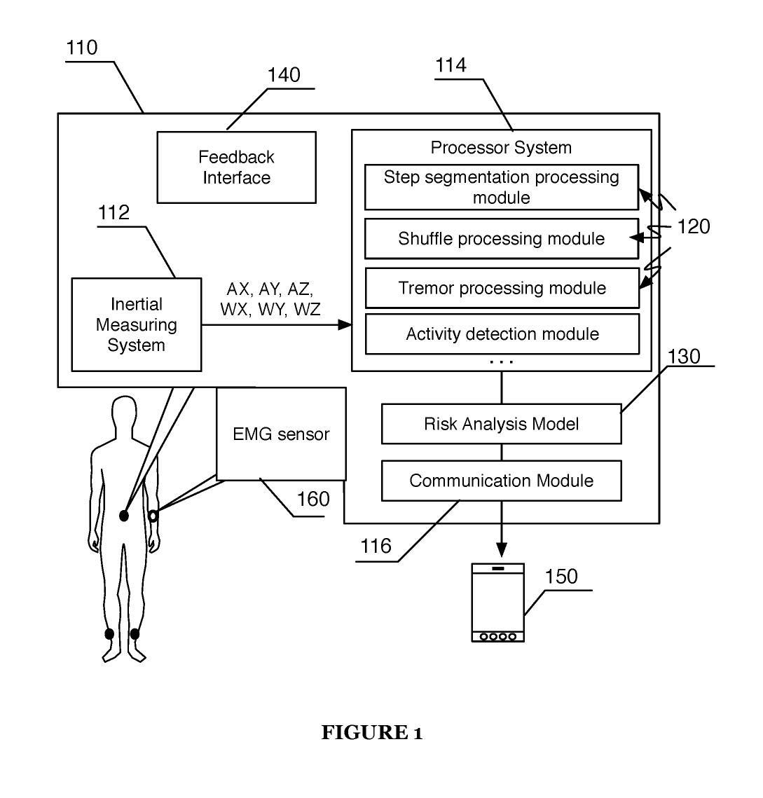

As shown in FIG. 1, a system for elderly fall prediction, detection, and prevention of a preferred embodiment can include a biomechanical sensing device 110, biomechanical processing modules for a set of gait metrics and activity classifiers 120, a risk analysis model 130, and at least one feedback interface 140. In one implementation, the system can include an application 150 communicatively coupled to that of the biomechanical sensing device 110. The biomechanical sensing device 110 and the application 150 can operate cooperatively in configured processing of collected kinematic data and generation of resulting interactions. The system may additionally include other biometric sensors 160 such as an electromyography (EMG), a temperature sensor, a heart rate sensor, and/or any suitable biometric sensor.

The system preferably converts sensor data from the biomechanical sensing device, in particular kinematic activity data such as accelerometer data or gyroscopic data, into detailed high-resolution mobility metrics based on biomechanical motion of the user. As one preferred application, the mobility metrics that are generated can be used to determine if a patient is slowly (over days, weeks, or months) degrading biomechanically to a point where they are at high risk of falling. The system can also detect when a patient has fallen and can characterize the falling motion before, during, and after the fall. If a fall is detected, an alert can go to the system and emergency responders or people listed as emergency contacts can be notified. Further, the system can monitor improvements in the mobility metrics in addition to or as an alternative to analyzing degradation or risks. This may be used after a user has a fall or injury where they want to improve their mobility.

The system is preferably part of a biomechanical sensing platform. The biomechanical sensing platform can include hardware, software algorithms, applications, and/or web services that can help solve new problems through tracking and analyzing high-resolution motion mechanics of a patient. The biomechanical sensing platform may additionally secure a cloud database that stores all user data that can be shared with the appropriate audiences such as a patient, nurse, physician, insurer, family, or the like. The biomechanical sensing platform may be used such that multiple instances of biomechanical sensing devices can be used for different users but with a shared biomechanical sensing platform. Alternatively, the system could be a standalone system that is not dependent on a shared cloud platform or other shared computing resources.

The system preferably includes at least one device component including the biomechanical sensing device 110 that is physically coupled to the body of the user. A biomechanical sensing device 110 of a preferred embodiment functions to collect kinematic data that is then transformed to a mobility metric. Depending on the specific workout activity, the device can be worn on the waist, pelvis, upper body, shoes, thigh, arms, wrists or head.

In some implementations, the biomechanical sensing device can be clipped onto the waist of a pair of shorts, magnetically attached, or slipped into a pocket of a garment, pehaps specially designed for the purpose. One or multiple devices can also be embedded into items commonly worn on the body, including but not limited to watches, wristbands, headbands, rings, necklaces, belts, back braces, bras, underwear, t-shirts, pants, shorts, yoga pants, wrist & arm bands, eyeglasses, handkerchiefs, hats, socks or shoes. Devices can also be attached to the body with adhesive patches. Depending on applications, the sensor can be worn on the waist, upper body, shoulders, spine, knee, shoes, thigh, ankles, shins, arms, wrists, neck or head. The device can also be embedded into any other form factor that lets the device sit securely on a user such as eyeglasses. The device can also be embedded into any other form factor that lets the device sit securely on a user.

The biomechanical sensing device 110 can include an inertial measurement unit 112, a processor 114, and optionally a communication module 116. The biomechanical sensing device 110 can additionally include any suitable components to support computational operation such as a processor, data storage, RAM, an EEPROM, user input elements (e.g., buttons, switches, capacitive sensors, touch screens, and the like), user output elements (e.g., status indicator lights, graphical display, speaker, audio jack, vibrational motor, and the like), communication components (e.g., Bluetooth LE, Zigbee, NFC, Wi-Fi, cellular data, and the like), and/or other suitable components.

The biomechanical sensing device 110 may serve as a standalone device where operation is fully contained in one device. The biomechanical sensing device 110 may additionally or alternatively communicate with at least one secondary system such as an application 150 operating on a computing device; a remote activity data platform (e.g., a cloud-hosted platform); a secondary device (e.g., a mobile phone, a smart watch, computer, TV, etc.); or any suitable external system.

In one variation, the system uses a multi-point sensing approach, wherein a set of inertial measurement units 112 measure motion at multiple points. The inertial measurement units 112 can be integrated into distinct devices wherein the system includes multiple communicatively coupled devices that can be mounted to different body locations. The points of measurement may be in the waist region, the upper leg, the lower leg, the foot, and/or any suitable location. Other points of measurement can include the upper body, the head, or portions of the arms. Various configurations of multi-point sensing can be used for sensing different mobility metrics. Different configurations may offer increased resolution, more robust sensing of one or more signals, and for detection of additional or alternative biomechanical signals. A foot biomechanical monitor variation could be attached to or embedded in a shoe. A shank or thigh biomechanics monitor could be strapped to the leg, embedded in an article of clothing, or positioned with any suitable approach. In a preferred implementation, the system includes a pelvic monitoring device that serves as a base sensor as many aspects of exercise activities can be interpreted from pelvic activity.

Multiple points of sensing can be used to obtain motion data that provides unique motion information that may be less prevalent or undetectable from just a single sensing point. Multiple points can be used in distinguishing alternative biomechanical aspects and/or to detect particular biomechanical attributes with more resolution or consistency. Multiple points may be used for detecting foot gait attributes, knee flex angle, and/or distinguishing between right and left leg or arm actions. Single point sensing may additionally be applied to right and left leg or arm attributes, upper core body or arms. The multiple points can be used to obtain clearer signals for particular actions such as when a user bends to pick up a heavy object or rotates his body left or right. Multiple points can additionally be used in providing relative kinematics between different points of the body. The relative angular orientation and displacement can be detected between the foot, thigh, pelvic, thoracic and neck regions. Similarly, relative velocities between a set of activity monitor systems can be used to generate particular mobility metrics.

The inertial measurement unit 112 functions to measure multiple kinematic properties. An inertial measurement unit 112 can include at least one accelerometer, gyroscope, magnetometer, and/or other suitable inertial sensor. The inertial measurement unit 112 preferably includes a set of sensors aligned for detection of kinematic properties along three perpendicular axes. In one preferred variation, the inertial measurement unit 112 is a 9-axis motion-tracking device that includes a 3-axis gyroscope, a 3-axis accelerometer, and a 3-axis magnetometer. The sensor device can additionally include an integrated processor that provides sensor fusion. Sensor fusion can combine kinematic data from the various sensors to reduce uncertainty. In this application, it may be used to estimate orientation with respect to gravity and may be used in separating forces or sensed dynamics from gravity. The on-device sensor fusion may provide other suitable sensor conveniences. Alternatively, multiple distinct sensors can be combined to provide a set of kinematic measurements.

An inertial measurement unit 112 and/or the biomechanical sensing device 110 can additionally include other sensors such as an altimeter, GPS, or any suitable sensor. Additionally, the system can include a communication channel via the communication module 116 to one or more computing devices with one or more sensors. For example, an inertial measurement unit 112 can include a Bluetooth communication channel to a smart phone, and the smart phone can track and retrieve data on geolocation, distance covered, elevation changes, land speed, topographical incline at current location, and/or other data.

The processor 114 functions to transform sensor data generated by the inertial measurement unit 112. The processor 114 can include a calibration module and a set of processing modules used in interpreting mobility metrics from the sensor data. The processing can take place on the biomechanical sensing device 110 or be wirelessly transmitted to a smartphone, computer, web server, and/or other computing system that processes the biomechanical signals.

The processor 114 used in applying signal processing on the sensor data can be integrated with the biomechanical sensing device 110. The processor 114 may alternatively be application logic operable on a secondary device such as a smart phone. In this variation, the processor 114 can be integrated with the user application 150. In yet another variation, the processor 114 can be a remote processor accessible over the network. A processor 114 on the biomechanics sensing device 110 or the application 150 can perform the biomechanics analysis in real-time or send the raw sensor data or partially processed data to a software application running on a smartphone, computer, home hub, web server or other computer medium for processing. Remote processing may enable large datasets to be more readily leveraged when analyzing kinematic data.

The communication module 116 functions to relay data between the biomechanical sensing device 110 and at least one other system. The communication module 116 may use Bluetooth, Wi-Fi, cellular data (e.g., 2G, 3G, 4G, and/or LTE telecommunication networks), and/or any suitable medium of communication. For example, the communication module 116 can be a Bluetooth chip with RF antenna built into the device. As discussed, the system may be a standalone device where there is no communication module 116.

The biomechanical sensing device 110 can additionally include one or more feedback elements, which function to provide a medium for delivering real-time feedback to the user. A feedback element can include a haptic feedback element (e.g., a vibrational motor), audio speakers, a display, or other mechanisms for delivering real-time feedback. Other user interface elements for input and/or output can additionally be incorporated into the device such as audio output elements, buttons, touch sensors, and the like.

In some variations, the system may include one or more biometric sensors 160. Preferably, the biometric sensor includes an electromyography (EMG) sensor, a pulse oximeter sensor, a temperature sensor, a galvanic skin response (GSR) sensor, and/or other suitable biometric sensors. Detection of electrical activity of the muscles can be used in interpreting muscle activity. Muscle activity combined with biometric modeling can be used to understand the effectiveness of an exercise and if the correct muscles are being activated properly.

The biomechanical processing modules 120 of the system function to characterize gait dynamics, a user activity graph, and/or other mobility metrics. A first set of biomechanical processing modules measure properties of gait locomotion (e.g., walking, running and the like). A second set of biomechanical processing modules may classify or detect various activity states. The time-based record of activity states can then be compiled into a user activity graph.

While walking or running, the biomechanical processing modules can quantify gait dynamics such as: step cadence (number of steps per minute); ground contact time; left or right foot stance time; forward/backward braking forces; upper body trunk lean; upper body posture; step duration; step length; swing time; step impact or shock; activity transition time; stride symmetry/asymmetry; left or right foot detection; pelvic dynamics (e.g., pelvic stability; range of motion in degrees of pelvic drop, tilt and rotation; vertical displacement/oscillation of the pelvis; and/or lateral displacement/oscillation of the pelvis); motion path; balance; turning velocity and peak velocity; foot pronation; vertical displacement of the foot; neck orientation; double stance time; tremor quantification, shuffle detection, and/or other suitable gait metrics.

The biomechanical processing modules related to the user activity graph may classify or detect in the sensor data various activity states. A user's activity graph can be a time series record of specific activities performed by the user, the amount of time performing that specific activity, and/or the biomechanical quality of the activity being performed. The biomechanical processing modules used for generating the user activity graph could be configured for: standing detection, lying down detection, in bed detection, sitting detection, walking detection, runing detection, car/biking/commute detection, and/or other forms of activity detection. In one variation, this may be represented as modules that track time spent performing the various detectable activities.

In some variations, activity states can relate to characteristics or biomechanical quality of an activity. For example, a subset of biomechanical processing modules could be configured for: limp detection, tremor detection, stumble detection, shuffle detection, double stance time tracking, fall detection, and the like.

The various mobility metrics and/or additional biomechanical processing modules may additionally assist in tracking other aspects relating to mobility such as tracking the number of walking and/or running steps, posture/orientation of the user during different activities, the number of calories that were burned during all of these activities, and/or other aspects.

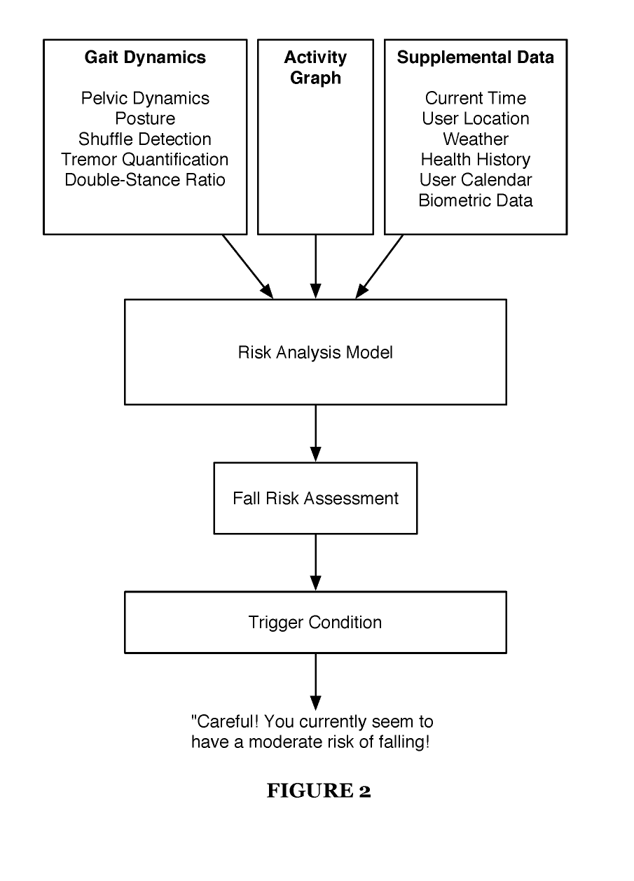

The risk analysis model 130 functions to process the mobility metrics and determining a fall risk assessment. The risk analysis model 130 preferably uses the mobility metrics as one source of data input. The risk analysis model 130 may additionally look at other data inputs such as weather, temperature, a user's location, a user's diet, a user's calendar events, home temperature data, other biometric sensor data such as heart rate, blood glucose levels, and the like as shown in FIG. 2.

The risk analysis model can preferably differentiate between different levels of risk. In an exemplary implementation, the risk analysis model may output three risk assessments: low risk, moderate risk, and high risk. The output could alternatively be any suitable type of measurement or set of classifications.

In one variation, the risk analysis model can generate a rest prediction, which is a generated recommendation for rest customized to the current situation. The rest prediction can be a time recommendation, but may additionally include a type of recommended rest (catching breath, sitting down, lying down, etc.).

The risk analysis model 130 in one variation can be configured for processing logic, rules, conditions, or other suitable heuristics used in assessing risk. As one example, sudden negative changes in mobility metrics can be indicators of moderate to high risk. As another example, particular patterns of mobility metrics or a recent history of different mobility events (e.g., excessive shuffling or double stance walking) could also be indicators of moderate to high risk.

The risk analysis model 130 could additionally be configured to alter or otherwise weight the risk analysis based on contributing factors such as time of day, location, and weather. In a time-based example, detected activity at night or odd hours may have more sensitive classification of risk. In a location-based example, detected activity in high-risk locations such as kitchens, bathrooms, and near stairs could trigger more sensitive classification of risk. In a temperature related example, the risk analysis could be more sensitive when the weather is hotter or colder.

The risk analysis model 130 can additionally or alternatively integrate machine learning models that use statistical or data driven modeling to the measuring and classification of fall risk. Preferably a machine learning model can be trained cooperatively a heuristically model as more data is collected and used for training.

A feedback interface 140 functions to provide some form of feedback to the user. The feedback interface 140 may be integrated with the biomechanical sensing device 110, the application 150, and/or any suitable device. The feedback interface is preferably activated in response to different mobility metrics and/or fall risk assessments. A feedback interface 140 preferably enables activation of one or more feedback outlets such as a display, an audio system, haptic feedback, and the like. In one variation, the system can enable optional use of an application 150.

The application 150 functions as one potential outlet for conveying information related to mobility metrics of a user. The application 150 is preferably used in combination with the biomechanical sensing device 110 to facilitate interactions with the user and/or coordinate processing and synchronization of data. The user application 150 can be any suitable type of user interface component. An application 150 is preferably user accessible on a personal computing device as a native application or as an internet application. Preferably, the user application 150 is a graphical user interface operable on a user computing device. The user computing device can be a smart phone, a desktop computer, a TV-based computing device, smart-home computing device, a wearable computing device (e.g., a watch, glasses, etc.), an audio computing assistant, a smart TV, and/or any suitable computing device. The user application 150 can alternatively be a website accessed through a client browsing device.

The application 150 may allow the user to sync data from the device, configure the device and settings, and view the data from the device. The application 150 may also process the raw signals from the device, provide feedback to the user and communicate with a remote data platform that can sync data, send firmware updates, or provide additional context such as social comparisons with other users to create a more compelling user experience.

In addition, the application 150 can connect to a cloud database of a data platform where user data can be uploaded. The uploaded data can then be analyzed to communicate mobility metrics, alerts, and/or other data to caretakers and/or medical personnel. In some variations, a customized application could similarly be provided for the caretakers and/or medical personnel. Data collected from multiple users could additionally be used in enhancing detection and modeling of the mobility metrics.

### 3. Method

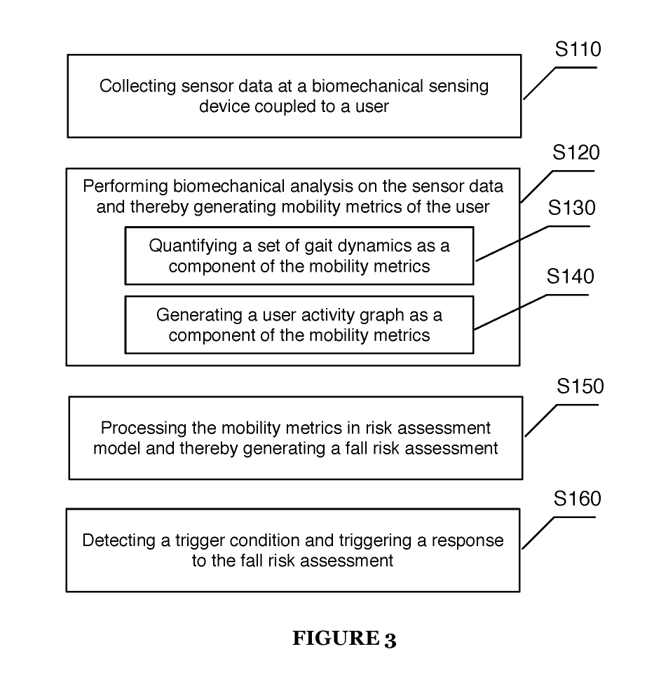

As shown in FIG. 3, a method for elderly fall prediction, detection, and prevention of a preferred embodiment can include collecting sensor data at a biomechanical sensing device coupled to a user Silo; performing biomechanical analysis on the sensor data and thereby generating mobility metrics of the user S120; processing the mobility metrics in risk assessment model and thereby generating a fall risk assessment S150; and detecting a trigger condition and triggering a response to the fall risk assessment S160. Performing biomechanical analysis on the sensor data preferably can include quantifying a set of gait dynamics as a component of the mobility metrics S130, and/or generating a user activity graph as a component of the mobility metrics S140.

The method is preferably implemented in connection with a biomechanical sensing platform and more specifically a biomechanical sensing device such as the ones described herein. However, the method may alternatively be implemented by any suitable system.

The method is preferably implemented in connection with a user that acts as a subject for mobility analysis. The method is preferably implemented periodically and/or continuously at different times. The biomechanical sensing device may exclusively operate in a mobility monitoring operating mode when implementing the method described herein. Alternatively, the biomechanical sensing device may selectively use the mobility monitoring operating mode at distinct times, operating in alternative modes at other instances.

Block S110, which includes collecting sensor data at a biomechanical sensing device coupled to a user, wherein the sensor data includes at least accelerometer data, functions to sense, detect, or otherwise obtain sensor data of the user. In particular, the sensor data includes kinematic data relating to motion and/or orientation of some portion of a user's body. The sensor data can additionally include collecting electromyography (EMG) data, temperature data, heart rate/pulse data, pulse oximeter sensor, a temperature sensor, skin electrical characteristics, respiratory rate, and/or other biometric data.

The biomechanical sensing device is preferably coupled to the user. Different variations may be designed for the biomechanical sensing device to be physically coupled at different locations. In one preferred implementation, the biomechanical sensing device is coupled to the user in the pelvic region. In another preferred implementation, the biomechanical sensing device or a secondary sensor of the device can be coupled to the user on one or both legs, in particular the foot. For example, an inertial measurement system element of the biomechanical sensing device could be attached or integrated into footwear worn by the user.

The kinematic data can be collected with an inertial measurement system that may include an accelerometer system, a gyroscope system, and/or a magnetometer. Preferably, the inertial measurement system includes a three-axis accelerometer and gyroscope. The kinematic data is preferably a stream of kinematic data collected over periods of time when a task is performed. The kinematic data may be collected continuously but may alternatively be selectively activated in response to different events.

In one variation, data of the kinematic data is raw, unprocessed sensor data as detected from a sensor device. Raw sensor data can be collected directly from the sensing device, but the raw sensor data may alternatively be collected from an intermediary data source. In another variation, the data can be pre-processed. For example, data can be filtered, error corrected, or otherwise transformed. In one variation, in-hardware sensor fusion is performed by an on-device processor of the inertial measurement unit. The kinematic data is preferably calibrated to some reference orientation. In one variation, automatic calibration may be used as described in U.S. patent application Ser. No. 15/454,514 filed on 9 Mar. 2017, which is hereby incorporated in its entirety by this reference.

Any suitable pre-processing may additionally be applied to the data during the method. In one variation, collecting kinematic data can include calibrating orientation and normalizing the kinematic data as part of the data collection process, the biomechanical analysis process, or any suitable process.

An individual kinematic data stream preferably corresponds to distinct kinematic measurements along a defined axis. The kinematic measurements are preferably along a set of orthonormal axes (e.g., an x, y, z coordinate plane). In some variations, the axis of measurements may not be physically restrained to be aligned with a preferred or assumed coordinate system of the activity. Accordingly, the axis of measurement by one or more sensor(s) may be calibrated for analysis by calibrating the orientation of the kinematic data stream. One, two, or all three axes may share some or all features of the calibration, or be calibrated independently. Alternatively, the sensor(s) used in acquiring the kinematic data (e.g., an inertial measurement unit) may have substantially consistent orientation when worn by a user, in which case no orientation or alternative orientation approaches may be used.

The kinematic measurements can include acceleration, velocity, displacement, force, rotational acceleration, rotational velocity, rotational displacement, torque, tilt/angle, and/or any suitable metric corresponding to a kinematic property of an activity. Preferably, a sensing device provides acceleration as detected by an accelerometer and angular velocity as detected by a gyroscope along three orthonormal axes. The set of kinematic data streams preferably includes acceleration in any orthonormal set of axes in three-dimensional space, herein denoted as x, y, z axes, and angular velocity about the x, y, and z axes. Additionally, the sensing device may detect magnetic field through a three-axis magnetometer.

Calibrating the kinematic data can involve standardizing the kinematic data and calibrating the kinematic data to a reference orientation such as a coordinate system of the participant. The nature of calibration can be customized depending on the task and/or kinematic activity. The device including the sensor(s) can be attached or otherwise fixed into a certain position during an activity. That position can be static during the activity but may also be perturbed and change, wherein recalibration may be performed again.

Block S120, which includes performing biomechanical analysis on the sensor data and thereby generating mobility metrics of the user, functions to transform sensor data of real world motion into modeling of various properties related to activities and performance of those activities by a user.

There are a number of indicators that lead to higher risk of falling that can be extracted through biomechanical analysis. Some potential mobility metrics can include pelvic instability which includes large pelvic drop and pelvic rotation values; stride asymmetries in pelvic drop, pelvic rotation, and ground contact time; lateral pelvis sway (rocking back and forth from left to right); low vertical displacement of feet (shuffling gait); high ratio of double stance time to single stance time, and sudden changes in body position. Other activity states and gait-based biomechanical metrics also may add significantly to quantifying the risk of a user who is about to fall. For example, upper body posture, neck posture, and trunk lean can also be indicators of fall risk.

Performing biomechanical analysis preferably includes quantifying a set of gait dynamics as a component of the mobility metrics S130 and generating a user activity graph as a component of the mobility metrics S140.

Block S130, which includes quantifying a set of gait dynamics as a component of the mobility metrics, functions to transform one or more elements of the kinematic data into biomechanical characterizations of static or locomotion-associated actions or states (generally referred to as biomechanical signals). The biomechanical signals are preferably measurements of some aspect relating to how the user moves their body when walking, running, or otherwise moving. This can additionally include detecting these attributes appropriately with movement assistance such as when using a walker, a cane, crutches, arm braces, and the like. The method can additionally include quantifying other biomechanical aspects that may not be exclusively associated with locomotion such as posture (e.g., when standing, sitting, or lying down) and tremor quantification.

In one variation, biomechanical signals may be generated in a manner substantially similar to that described in U.S. patent application Ser. No. 15/283,016, filed 30 Sep. 2016, which is hereby incorporated in its entirety by this reference.

Generating locomotion biomechanical measurements can be based on step-wise windows of the kinematic data—looking at single steps, consecutive steps, or a sequence of steps. In one variation, generating locomotion biomechanical measurements and more specifically gait biomechanical measurements can include generating a set of stride-based biomechanical signals comprising segmenting kinematic data by steps and for at least a subset of the stride-based biomechanical signals generating a biomechanical measurement based on step biomechanical properties. Segmenting can be performed for walking and/or running. In one variation steps can be segmented and counted according to threshold or zero crossings of vertical velocity. A preferred approach, however, includes counting vertical velocity extrema. Another preferred approach includes counting extrema exceeding a minimum amplitude requirement in the filtered, three-dimensional acceleration magnitude as measured by the sensor. Another preferred approach may count segments by identifying threshold crossings or extrema in vertical acceleration followed by identification of subsequent plateau regions in vertical velocity of relatively constant value or other specific criteria. Requiring two or more conditions to be satisfied to count segments may improve accuracy of the segmentation when the input waveforms are predominantly non-periodic or noisy. Different approaches may be used in different conditions. For example, the multiple condition operation mode described above may be activated when a primary error correcting detection mode is unavailable (e.g., causing errors, satisfying a poor signal condition, etc.).

The set of stride-based biomechanical signals can include step cadence (number of steps per minute); ground contact time; left or right foot stance time; double stance time, forward/backward braking forces; upper body trunk lean; upper body posture; step duration; step length; swing time; step impact or shock; activity transition time; stride symmetry/asymmetry; left or right foot detection; pelvic dynamics (e.g., pelvic stability; range of motion in degrees of pelvic drop, tilt and rotation; vertical displacement/oscillation of the pelvis; and/or lateral displacement/oscillation of the pelvis); motion path; balance; turning velocity and peak velocity; foot pronation; vertical displacement of the foot; neck orientation; tremor quantification, shuffle detection, and/or other suitable gait or biomechanical metrics.

Cadence can be characterized as the step rate of the participant.

Ground contact time is a measure of how long a foot is in contact with the ground during a step. The ground contact time can be a time duration, a percent or ratio of ground contact compared to the step duration, a comparison of right and left ground contact time (e.g., a variation of an asymmetry metric) and/or any suitable characterization.

Braking or the intra-step change in forward velocity is the change in the deceleration in the direction of motion that occurs on ground contact. In one variation, braking is characterized as the difference between the minimum velocity and maximum velocity within a step, or the difference between the minimum velocity and the average velocity within a step. Braking can alternatively be characterized as the difference between the minimal velocity point and the average difference between the maximum and minimum velocity. A step impact signal may be a characterization of the timing and/or properties relating to the dynamics of a foot contacting the ground.

Upper body trunk lean is a characterization of the amount a user leans forward, backward, left or right when walking, running, sitting, standing, or during any suitable activity. More generally, upper body posture could be measured or classified in a number of ways.

Step duration is the amount of time to take one step. Stride duration could similarly be used, wherein a stride includes two consecutive steps.

Step length is the forward displacement of each foot. Stride length is the forward displacement of two consecutive steps of the right and left foot.

Swing time is the amount of time each foot is in the air. Ground contact time is the amount of time the foot is in contact with the ground.

Step impact is the measure of the force or intensity of contact with the ground in a vertical direction during ground contact. It could be measured as a force, a deceleration rate, or other similar metric.

Activity transition time preferably characterizes the time between different activities such as lying down, sitting, standing, walking, and the like. A sit-to-stand transition is the amount of time it takes to transition from a sitting state to a standing state.

Left and right step detection can function to detect individual steps. Any of the biomechanical measurements could additionally be characterized for left and right sides.

Stride asymmetry can be a measure of imbalances between different steps. It quantifies the difference between left-side gait mechanics and right-side gait mechanics. Strides or bouts of strides can be identified as symmetrical or asymmetric for each relevant gait component. The asymmetric components could be aggregated overtime wherein asymmetry patterns of a stride that were exhibited over an extended duration could be reported. Temporary non-consistent asymmetries in a stride may be left as unreported since they may be normal responses to the environment. Asymmetric gait dynamics preferably describe asymmetries between right and left steps. It can account for various factors such as stride length, step duration, pelvic rotation, pelvic drop, ground contact time, and/or other factors. In one implementation, it can be characterized as a ratio or side bias where zero may represent balanced symmetry and a negative value or a positive value may represent left and right biases respectively. Symmetry could additionally be measured for different activities such as posture asymmetry (degree of leaning to one or another side) when standing.

For step length asymmetries, detecting segments of the sensor data with asymmetric gait dynamics comprises detecting right and left step lengths and comparing the right step length(s) and left step length(s). The comparison, which can be the difference between the lengths (or some average of lengths), or ratio of lengths (or average lengths) may be used as the measure of asymmetry. For example, a value near zero indicates step lengths are similar or the same in length and a large value indicates a larger discrepancy. In another example, a ratio close to 1 is symmetrical, whereas values greater than or less than 1 (such as 1.2) may indicate asymmetry. The comparison can be normalized for user height and/or the step length of the greater length or to the specified foot (e.g., a right foot). In one variation, the asymmetric step length conditions could be classified when the comparison satisfies some condition (e.g., being greater than a step length difference or ratio threshold). Significant step length asymmetries may be indicators of a limp, dragging of a leg, localized pain/weakness in a leg, or other symptoms. Different conditions based on stride asymmetry can be used to determine when to deliver feedback or initiate another response. Sudden changes in stride asymmetry in particular can be a condition used to trigger an alert.

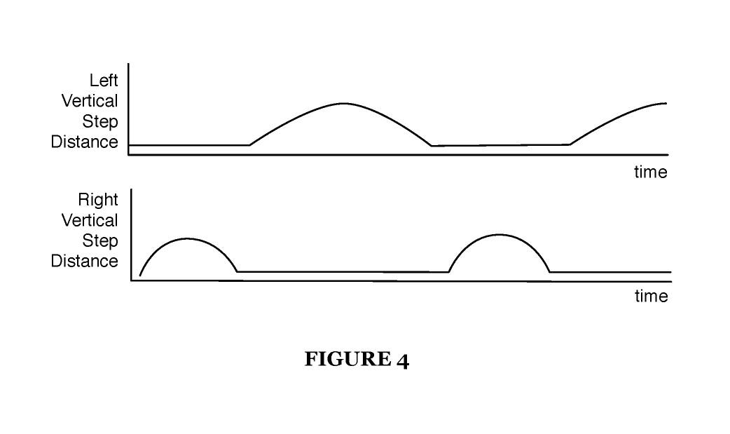

For step time differences, detecting segments of the sensor data with asymmetric gait dynamics can be substantially similar to step lengths except that the step time for left and right steps can be compared as shown in FIG. 4.

For pelvic tilt or posture asymmetries, detecting asymmetric gait dynamics can include detecting orientation states during a right step and orientation states during a left step and comparing the right and left orientation states. Here orientation states can include pelvic dynamics (e.g., how they lean over the hip)

Pelvic dynamics can be represented in several different biomechanical signals including pelvic rotation, pelvic tilt, and pelvic drop. Pelvic rotation (i.e., yaw) can characterize the rotation in the transverse plane (i.e., rotation about a vertical axis). Pelvic tilt (i.e., pitch) can be characterized as rotation in the sagittal plane (i.e., rotation about a lateral axis). Pelvic drop (i.e., roll) can be characterized as rotation in the coronal plane (i.e., rotation about the forward-backward axis).

Vertical oscillation of the pelvis is characterization of the up and down bounce during a step (e.g., the bounce of a step).

Lateral oscillation of the pelvis is the characterization of the side-to-side displacement during a stride possibly represented as a lateral displacement.

The motion path can be a position over time map for at least one point. Participants will generally have movement patterns that are unique and generally consistent between activities with similar conditions.

Balance can be a measure of posture or motion stability when walking, running, standing, carrying, or performing any suitable activity.

Turn speed can characterize properties relating to turns by a user. In one variation, turn speed can be the amount of time to turn. Additionally or alternatively turn speed can be characterized by peak velocity of turn, and/or average velocity of turn when a user makes a turn in their gait cycle.

Foot pronation could be a characterization of the angle of a foot during a stride or at some point of a stride. Similarly foot contact angle can be the amount of rotation in the foot on ground contact. Foot impact is the upward deceleration that is experienced occurring during ground contact. The body-loading ratio can be used in classifying heel, midfoot, and forefoot strikers. The foot lift can be the vertical displacement of each foot. The motion path can be a position over time map for at least one point of the user's body. The position is preferably measured relative to the user. The position can be measured in one, two, or three dimensions. As a feature, the motion path can be characterized by different parameters such as consistency, range of motion in various directions, and other suitable properties. In another variation, a motion path can be compared based on its shape.

The foot lift can be the vertical displacement of each foot.

Neck tilt can be the posture or orientation of the head. Neck orientation can include neck/head tilt (i.e., pitch—rotation in the sagittal plan), neck/head roll (i.e., rotation about the forward-backward axis), and head neck rotation (i.e., yaw—rotation in the transverse plane/rotation about a vertical axis).

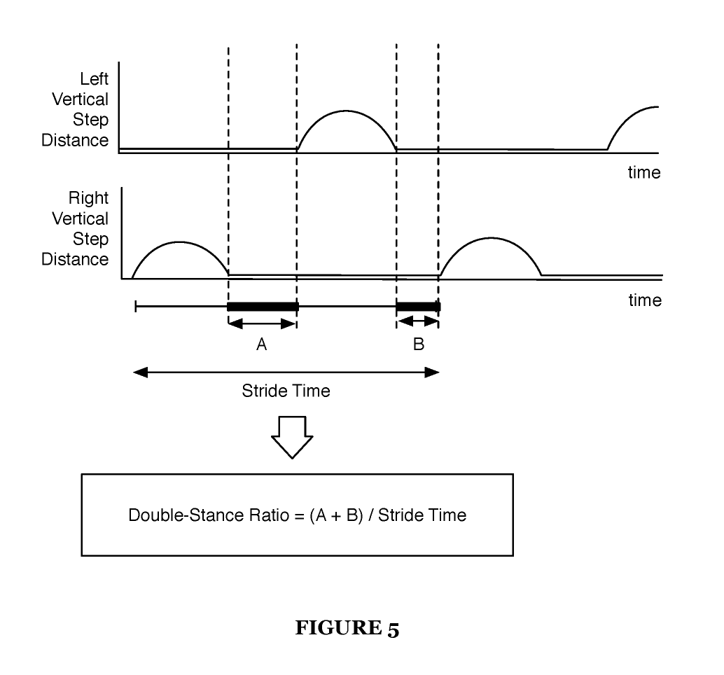

Double-stance time is the amount of time both feet are simultaneously on the ground during a walking gait cycle. Detecting segments of sensor data indicative of double stance gait patterns can include detecting a double stance condition in the ground contact time of the right and left steps as shown in FIG. 5. Double stance time is preferably detected and collected by detecting ground contact time for both feet and counting simultaneous foot contact time for the two feet. The duration of double stance time compared to the non-double stance time of a stride or step (i.e., double stance “duty cycle”) can be used as an indicator of poor mobility because the user is relying on keeping both feet on the ground. Users that are unstable on their feet may have a tendency to walk in a way that minimizes the amount of time they stand on one foot. Double stance time can also be represented by the ratio of an average double stance ground contact time to an average single stance ground contact time.

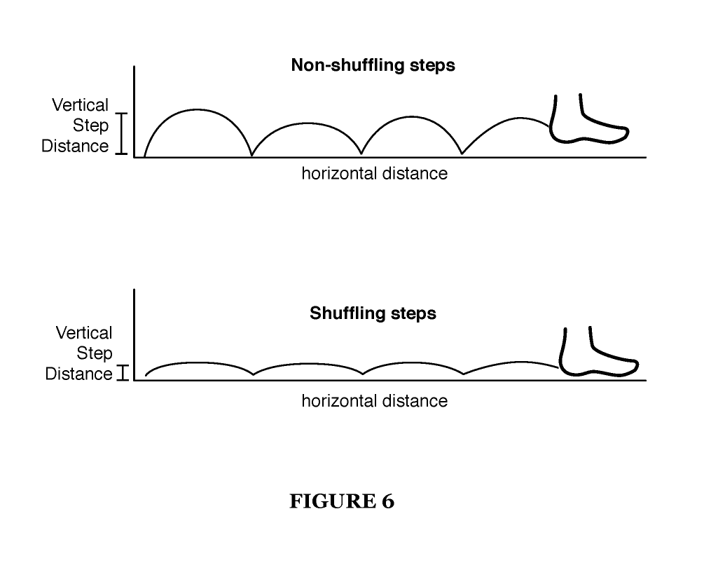

Shuffle detection can be a characterization of shuffling gate when moving. Shuffling may be a walking motion that lacks the vertical displacement of the feet when walking. In extreme cases this may be where a user doesn't lift their feet when walking and instead slides them across the floor. Accordingly, detecting segments of sensor data indicative of shuffling gait patterns can include detecting vertical step displacements of the right and/or left steps and classifying the gait as shuffling when vertical step displacements satisfy a shuffle condition as shown in FIG. 6. The shuffle detection may be based on vertical displacements that are below some step displacement threshold. The threshold and/or the measured vertical displacements can be normalized or otherwise adjusted to account for user height, age, and/or other factors. The shuffle condition may additionally look at vertical displacements over a particular time window. For example, an average vertical displacement of the past 1 minute of walking or average of the last 10 steps that is under the shuffle threshold may alternatively be a shuffle condition. The shuffle condition may also look at percentage of shuffling time for a stretch of walking. For example, walking short distances (e.g., when moving from point to point in the house) may be counted in one way while walking long distances (e.g., when walking long stretches of distance) may be counted another way. Individualized tracking and analysis for different types of walking paths can be performed for any suitable mobility metric.

Tremor quantification can include detecting tremors but can additionally be used in measuring duration, frequency response components, and magnitude of tremors. Detecting tremors preferably includes detecting vibrations or small vibrations within a certain frequency range and intensity range. In some cases, a tremor activity by a patient may have a frequency response such as between 4 Hz and 10 Hz, which could be characterized by the frequency response components. Additionally, range of motion may also quantify the tremor magnitude. Tremor detection can be isolated to particular parts of a stride or motion.

Biomechanical signals or gait dynamics may be expressed as variability or consistency metrics. Biomechanics variability or consistency can characterize variability or consistency of a biomechanical property such as of the biomechanical measurements discussed herein. The cadence variability may be one exemplary type of biomechanical variability signal, but any suitable biomechanical property could be analyzed from a variability perspective. Cadence variability may represent some measure of the amount of variation in the steps of the wearer. In one example, the cadence variability is represented as a range of cadences. The cadence variability may be used for interpreting the variations in walking patterns

Measuring posture functions to generate a metric that reflects the nature of a user's posture and ergonomics. This is preferably performed when standing, walking, or running. Posture or position can additionally be used when sitting or lying down.

In one variation, measuring posture can be an offset measurement of the calibrated biomechanical sensing device orientation relative to a target posture orientation. A target posture orientation may be pre-configured. For example, an activity monitoring system with a substantially consistent orientation when used by a user may have a preconfigured target posture orientation. Alternatively, a target posture orientation may be calibrated during use automatically. Target posture orientation may be calibrated automatically upon detecting a calibration state. A calibration state may be pre-trained kinematic data patterns that signal some understood orientation. For example, sitting down or standing up may act as a calibration state from which calibration can be performed. A target posture orientation may alternatively be manually set. For example, a user may position their body in a desired posture orientation and select an option to set the current orientation as a target orientation. In another variation, the target orientation may change depending on the current activity. Accordingly, measuring posture can include detecting a current activity through the kinematic data (or other sources), selecting a current target posture orientation for the current activity and measuring orientation relative to the current target posture orientation.

In one variation, measuring posture may include characterizing posture. Characterizing posture may not generate a distinct measurement, and instead classifies different kinematic states in terms of posture descriptors such as great posture, good posture, bad posture, and dangerous posture. Various heuristics and/or machine learning may be applied in defining classifications and detecting gesture classifications.

Block S140, which includes generating a user activity graph as a component of the mobility metrics, functions to classify various activities of the user. Detecting a current physical activity state preferably includes analyzing kinematic data and detecting physical activity state from patterns in the kinematic data. Examples of detectable physical activity states can include driving, standing, sitting (e.g., sitting in a couch, sitting at a desk, and the like), striding (e.g., walking, running, jogging, and the like), lying down, and the like.

The activity graph is a data model that characterizes activity states over a period of time. From a data reporting standpoint, the activity graph can be used for reporting activity trends. This can be informational, but can also be used in promoting healthy ways of increasing or maintaining mobility.

Preferably, the activity graph can enable the history of activity over the course of a day to be analyzed. The activity graph can be particularly helpful in understanding the context of different mobility metrics. Previous activities, the duration of those activities, and the mobility metrics during those activities can be factored into assessing fall risk. Prior to having a significant rest period (e.g., sleep at night or a long nap), previous activity may be contributing factors that can increase the risk of a fall if it could lead to an overly fatigued state. Understanding activity-induced fatigue can additionally be used to determine when increased fall risk can be addressed by the user (e.g., a user can be coached to rest and regain strength before continuing activities) and/or when increased fall risk is a serious issue that should be addressed by a caretaker (e.g., if stumbling, and poor mobility metrics cannot be explained by general fatigue).

Different activities may have different mobility metrics that are tracked. For example, walking and/or running biomechanical signals can be collected only during walking and running activities. The analysis of the mobility metrics can additionally depend on the current detected activity. For example, lying, sitting, and standing states may each have different posture conditions that can be monitored. Block S150, which includes processing the mobility metrics in risk assessment model and thereby generating a fall risk assessment, functions to characterize mobility as it relates to mobility quality and risks. As one preferred objective, the method can be used to prevent or mitigate the risks associated with falls. One primary way the method addresses fall risk is in taking proactive mitigating actions. The processing of the mobility metrics preferably performs real-time and historical analysis on mobility metrics to determine when the mobility of a user transitions to a different level of risk. The fall risk assessment can be a score relating to the risk of a fall based on a general health state, which may be more long-term health analysis. For example, the fall risk assessment may change a fall risk score on a daily basis. The fall risk assessment may additionally or alternatively generate a more immediate score that provides a score related to the risk of a fall in substantially real time (e.g., updated hourly, every 1-3 minute, within the 1-15 seconds, etc.). For example, the fall risk score could change as the user goes about their day, goes to different locations, and performs different activities.

There are a number of indicators that lead to higher risk of falling that the biomechanical sensing device monitors. Some indicators can include pelvic instability, which includes large pelvic drop (e.g., pelvic coronal drop) and pelvic rotation values, stride asymmetries in pelvic drop, pelvic rotation, and/or ground contact time. Lateral pelvis sway (rocking back and forth from left to right); shuffling gait; low vertical displacement of feet; sudden changes in body position, activity state and walking mechanics; and/or other movement properties may add significantly to quantifying the risk of a user who is about to fall. Additionally, upper body posture, neck posture and trunk lean can also increase fall risks.

When a user is exhibiting some or all of these biomechanical indicators, over a certain amount of time, the system may increase the risk profile or automatically label the individual as high risk. For instance, if a user was inadvertently walking with a large upper body trunk lean, the user may lose balance and fall over, hence the system and device can assign a higher risk score.

Additionally, if a user exhibits sudden changes in movement behavior or walking gait, or exhibits abnormal gait such as a limp or stumble, the device will detect these events and increase the risk profile of the individual. The system can take in multiple inputs, including the ones described above to determine the risk profile of an individual. Once the system has determined the user to be high risk, it will alert the nursing staff, emergency contact, etc. to take action. In addition, the method can alert the user in real-time. For example, the system and device could provide haptic feedback, voice/audio feedback or visual feedback in real-time to remind the user to be careful. Alerts and notifications can similarly be generated and communicated to appropriate caretakers or systems. Other suitable actions could alternatively be initiated as part of Block S160.

In one variation, shuffle detection, double-stance time, and tremors are mobility metrics that can be indicators of poor mobility and increased risk of falling. In particular, processing the mobility metrics in a risk assessment model can include increasing the risk of a fall in the fall risk assessment with detected increases in duration of shuffle-associated strides, double-stance time, and/or the amount of tremors.

As one implementation variation, a heuristic or a condition associated with a risk of falling can depend at least partially on shuffle detection. When a shuffle detection metric satisfies some shuffle condition, the trigger condition of block S160 may be satisfied triggering a response. The shuffle condition can be based on the duration of shuffling, the ratio of shuffling during movement, or other shuffle related characterizations. For a user able to walk without shuffling at times, triggering a response may be conditioned on when the onset of shuffling indicates fatigue, pain, stiffness, or other contributing factors to temporary poor mobility quality. A response could be generated with the intent to caution the user of temporary lapse in mobility quality. For a user with sustained shuffling gait, the degree of shuffling may be used for triggering an alert. Alternatively, the user could be notified when they begin moving with improved mobility quality, which may function to encourage a user.

As another implementation variation, a heuristic or a condition associated with a risk of falling can depend at least partially on double-stance time. Double-stance time may indicate unsteadiness when moving as the user must temporarily balance on both feet. Similar to shuffle detection, a trigger condition of block S160 may be satisfied that triggers a response when a shuffle detection metric satisfies some shuffle condition.

As another implementation variation, a heuristic or a condition associated with a risk of falling can depend at least partially on tremor detection. Tremor detection preferably is a measurement on some scale. When a tremor metric satisfies some tremor threshold condition, the trigger condition of block S160 may be satisfied triggering a response. The tremor threshold condition is preferably when the measurement of tremor activity exceeds some threshold, which functions to indicate that the number of tremors, duration of tremors, and/or intensity of tremors indicates a state of mobility quality that puts the user at risk.

In one variation, the method can include collecting supplemental data relating to the context of activities. The supplemental data can be an input to the risk assessment model and preferably augments the analysis of the mobility metrics. This can include collecting user location information, the current time of day, weather, temperature, environmental brightness, calendar activities, user diet, user drug dosage or medical treatments (physical therapy records, etc.), and/or other suitable information. For instance, there may be a particular location where the user has nearly tripped that can be recorded, as well as the times the user has nearly fallen or has fallen down. Additionally, a user who wakes up in the middle of the night to go to the bathroom, may be at higher risk during the period the individual is walking to/from the bathroom.

In one exemplary variation, the method includes collecting location data of the user and wherein the fall risk assessment is based in part on the location. The fall risk assessment can be weighted differently for different locations. For example, a real-time fall risk assessment may be more sensitive to mobility metrics when located in high risk locations. High risk locations can be locations within the home like the bathroom, stairs, the kitchen, and/or other dangerous locations. High risk locations can additionally be based on familiarity so locations commonly visited by a user would be assessed with greater tolerance in the fall risk assessment because the user is presumed to be more comfortable and familiar with the space. Locations infrequently visited or never previously visited by a user could be assessed with lower tolerance in the fall risk assessment because the user may be at greater risk of a fall since they are unfamiliar with the space. Location data can be collected via a GPS sensor, location service of a computing device, Wi-Fi or RFID location tracking, or other location tracking systems.

In another exemplary variation, current time is an input to the risk analysis model, wherein the analysis of the mobility metrics is weighted differently at different times of day. In particular, the mobility metrics can be analyzed in one mode during a first period of time (e.g., day hours) and analyzed in a second mode during a second period of time (e.g., night hours). Risk analysis modeling biased by the time of day functions to account for typical factors such as wakefulness, fatigue level, general visibility of the surrounding space, and/or other factors. In a similar variation, environmental brightness detection can account for visibility and similarly be used to augment the processing of mobility metrics in the risk assessment model.

In another exemplary variation, the method includes collecting weather data in proximity to the user; wherein the processing of mobility metrics in the risk assessment model is augmented by the weather data. For example, a first set of weather conditions can alter the fall risk assessment of mobility metrics negatively as compared to the fall risk assessment of the mobility metrics during a second set of weather conditions. In particular, the weather conditions include temperature (outside and/or inside). Colder temperatures or excessively hot temperatures may increase the risk of a fall for a given set of mobility metrics. For example, temperatures outside of the range 65°-80° could cause more sensitive analysis of the mobility metrics. Weather conditions like rain, snow, and wind could similarly negatively impact the fall risk assessment and be used in guiding a user to be more cautious. Weather may also impact the amount of rest needed and/or guidance recommendations. For example, on days with temperatures above 85° may alter recommended amounts of activities and/or prompt increased drinking of fluids.

In some variations, the method may include collecting user medical condition information that is used to set the conditions for analysis and monitoring. For example, the medical condition information of a user may indicate the user suffers or has suffered from affliction that impaired mobility of the right leg. The method could then monitor stride asymmetry that favors the right leg (e.g., short steps with the right leg) with stricter conditions (i.e., be more quick to provide feedback when the user starts favoring the right leg).

Additionally, the method may include user biometric data such as electromyography (EMG) data, a temperature data, heart rate/pulse data, pulse oximeter sensor, a temperature sensor, skin electrical characteristics, respiratory rate, and/or other biometric data as an input to the risk assessment model. Elevated biometric levels or other patterns can similarly be used to indicate increased risk.

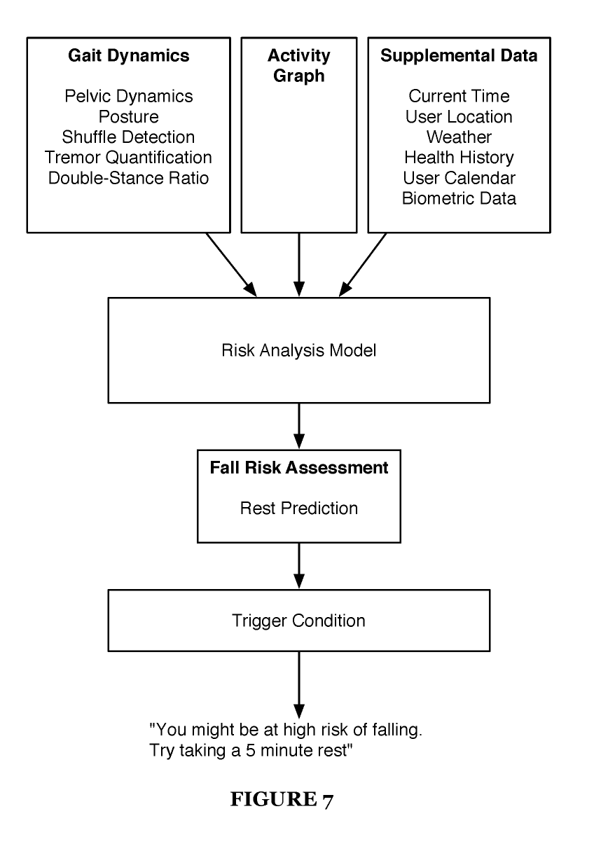

In one variation, the fall risk assessment is or includes a rest prediction metric, which functions to provide a recommendation of rest to address or mitigate current risks of a fall as shown in FIG. 7. The rest prediction metric preferably accounts for severity of the risk of a fall, current or typical mobility metrics of the user, and/or recent activity. The rest prediction metric is preferably scaled to recommend an appropriate amount and variety of rest that lowers the risk of a fall in a minimally invasive manner. For example, small spikes in fall risk caused by over exertion may generate a rest prediction for a brief 5 minute rest. Chronic degeneration of mobility detected at the beginning of the day may generate rest prediction to minimize mobility and increase risk for the next several hours or even over the course of the day.

Generating a rest prediction metric is preferably additionally accompanied by prompting the user to rest according to the rest prediction metric at appropriate moments in block S160. The rest recommendation is preferably provided to the user through a suitable feedback interface (e.g., a displayed graphic, a notification, an audio alert, etc.). The rest recommendation is preferably delivered when the mobility metrics satisfy some condition such as the risk of a fall increasing beyond some threshold.

The rest prediction metric could be a recommended amount of time for rest. In one preferred implementation, the generation of a rest prediction metric is accompanied by prompting the user to rest for an amount of time based on or specified by the rest prediction metric. The rest prediction metric could additionally or alternatively be a recommended variety of rest such as stopping motion and catching breath, sitting down, lying down, napping, and the like.

Other recommendations to mitigate falls can include time-based recommendations, location recommendations, weather-associated recommendations, and/or other suitable types of recommendation. These can similarly be used to guide a user in avoiding activities in more risky locations, weather conditions, or times of day.

In one variation, the risk assessment model can include or be a machine learning prediction model. The machine learning model preferably uses the mobility metrics as at least a subset of input features. The high resolution biomechanical data generated by the biomechanical sensing device along with location data, time, weather, temperature and other data sets can also be analyzed with machine learning models to help identify specific conditions, behaviors or patterns that predict the risk profiles of individuals that may be at high risk to falling.

A first approach is to use a population classification model based on labeled data of high-risk and non-high-risk states. The high-risk data set can be labeled and continually learn with additional input from the fall detection algorithm. When a fall is detected, the high risk state prior to the fall will be labeled. In addition, if a stumble or a near-fall event is detected, the data before the event is labeled. Accordingly, the method may include receiving a fall event report and updating a machine learning model with mobility metrics associated with the fall event report. The fall event report preferably includes the time of day and a descriptor of the event (e.g., a stumble, tripping on object, collapsing fall, critical fall, etc.). The mobility metrics associated with a fall event report can include mobility metrics at the time of the event and optionally mobility metrics leading up to the event (e.g., mobility metrics from earlier in that day, in the 30 minutes prior to the event, etc.).

The fall event reports may be submitted by the user. For example, a user application could be used by the user to report qualitative assessment of their day. Fall event reports could additionally or alternatively be reported by a caregiver. The application could enable reporting of particular incidents and their timing (e.g., reporting falls, stumbles, feelings of unsteadiness, and the like). The location could additionally be reported, but location may alternatively be determined based on sensed location at the time of the event. In some cases, qualitative assessment of a patient's day could be reported by rating their steadiness, energy levels, or other feelings for a particular time period, typically the current day. The application could additionally use the machine learning prediction model to attempt to classify events and then request reporting on those suspected events.

For example, the machine learning model may detect a possible stumble event when a user is on a walk. At the end of the day, the application could prompt the user to report on that suspected event. The user may indicate that there was no such event or possibly provide additional details if the user did notice the event. In some variations, such event reporting could be requested in substantially real-time. For example, a suspected event could be detected, and, subsequent to that event, a user application could trigger a notification like “It seems that you may have stumbled? If so please provide the following details . . . ”. This can function to highlight possibly issues of mobility quality to the user and to collect qualitative data.

The machine learning model preferably trains on the collected data. Event labeling of a population of users can be used for a general event detection model. Additionally, usage by a user can promote customized modeling for that particular user. Different machine learning models may also be used for different classes of users. Models could be targeted for particular age groups, affliction affiliation, movement patterns, and/or other user groupings.

Specific machine learning approaches include multi-layer neural networks, support vector machines, Bayes Nets, and deep learning networks to identify common characteristics of falling-risk based on the population. By using a supervised machine learning algorithm on the population, the algorithm can generalize to new individuals and new behaviors.

Another approach is to use an unsupervised clustering algorithm to find groups of data that are most dissimilar. These approaches include k-means, expectation-maximization algorithms, density-based clustering, principal component analysis, and auto-encoding deep learning networks to identify different states, which would correspond to high-risk walking or movement mechanics and low-risk walking and motion mechanics. By using an unsupervised learning algorithm, the model can find natural boundaries between the types of states.

The quality of movement can be quantified and stored throughout the entire day and uploaded in real-time or periodically to a software application for the user, their nurse, physician or emergency contacts to review.

Machine learning or other data-driven modeling can enhance the detection of fall risk. However, acquiring of such data in a way is challenging if there is no benefit to the user before a critical mass of data is collected. Accordingly, one implementation of the method transitions from a set of fall risk heuristics as described herein to increased reliance on machine learning models. This can function to provide tangible fall mitigation for early users and improved fall prediction and mitigation with increased usage.

Block S160, which includes detecting a trigger condition and triggering a response to the fall risk assessment, functions to use the fall risk assessment and/or mobility metrics in an action. The response can be used in a variety of ways. Preferably, this can include detecting a trigger condition of elevated risk as indicated in the fall risk assessment and triggering a response. The response can be user feedback (e.g., alerting the user of the current risk of falling) or communicating an alert or report to a caretaker. Transmitting an alert to a caretaker may be used for general reporting and/or for surfacing news of events and mobility data worth review by the caretaker.

When a user is labeled high risk as indicated by the fall risk assessment, the system can send an alert to the user's emergency contact or nurse. The system may automatically set up a teleconference with the user that day to check in with the user. During this time, the user can run through the normal movement routines, and the biomechanical sensing device can record and transmit all this data to the physician in real-time.

The system through audio, visual, and/or haptic feedback may communicate friendly reminders to be careful as they are at a higher risk of falling or suggest specific recommendations to the user such as drinking water to stay hydrated as many falls occur due to dehydration.

As described above, detecting a trigger condition and triggering a response can include prompting a user to rest. This may include communicating an alert that suggests user to use a walker, sit down, stretch, or add specific muscle strengthening exercises to a future exercise workout for the user. The prompt is preferably communicated or otherwise delivered through a suitable feedback interface. The nature of the rest may be based on the rest prediction metric described above.

The system could also provide the data and recommendations to on-call nurses, physicians, family members, call centers, a virtual AI health coach, and/or other suitable people or systems. These caretakers or systems could contact or otherwise engage with the user. For example, a phone call, video conference, or message could be delivered to the user and used to help guide the user in ways that mitigate the risk of falling. For example, in a nursing home or hospital, the method may alert an active nurse and establish an intercom session with the room of the user to see if they need any assistance.

For nursing homes or hospitals that have elder patients who are labeled high risk by the system or manually by the nurse, the system can provide additional notifications and alerts to help the nurses monitor the movement patterns of at-risk individuals. Preferably, triggering a response can include reporting incidents such as fall events, stumble events, tremor time, double stance time, particular fall risk assessment states, and/or other suitable mobility related information.

For example, if the patient is required to stay in bed, the system can detect if the patient transitions from a lying down position to a sitting position while in bed. The system and device can also detect if a patient transitions to higher risk sitting positions such as sitting on the edge of the bed. If a patient stands up, gets out of bed, or walks around, etc. the system can detect this and provide additional notifications and alerts to the user, nursing staff or management system.

If unwarranted user motion is detected, the nursing staff can be notified. The system can also remind the at-risk individual to stop walking around and sit down or get back into bed.

The nature of the reporting can additionally be selective in nature and in recipient. In one implementation, the method can be used to automatically report mobility metrics to the user, medical staff, emergency care point of contacts, and permitted family/friends. Basic mobility metrics and fall risk assessment information that warrants little attention may be reported in an application or web dashboard used by the user and possible medical staff or other entities. Fall risk assessments or other data of moderate concern may be sent as a notification to the user or medical staff with the aim of having the information seen or reviewed around the time of occurrence. Severe mobility related events (e.g., mobility metrics and/or fall risk assessment that satisfies a severity condition) such as a detected fall can automatically trigger an emergency communication to the appropriate destinations (e.g., emergency medical staff).

A web dashboard will be provided to allow the nurse to see all relevant movement data for an individual person or a group of persons. Adult children can also monitor their parents through the same web dashboard, smart phone app or similar computing device.

In the event that a fall has occurred as indicated by the method or indicated by user input, the motions before the fall, during and after the fall can be analyzed. The motions beforehand can be analyzed or labeled as high risk to help improve the system's high risk prediction models. The location and biomechanical data before the fall is analyzed and added to the prediction and prevention models. If a user falls multiple times, the system and data may help the care provider to identify the source of the fall. For example, if the falls happen at a particular time, location, and if the user has a particular walking gait, the system can refine the fall prediction. The system can also notify the care provider of a specific location where the user shuffles or nearly falls. The care provider can alter the high risk area to be more ergonomic.

The falling speed, impact, vertical distance, vertical and lateral accelerations, velocities and displacement motion paths, sensor orientation changes and speed of change, and other characteristics can be measured to detect and analyze a fall occurrence. False positives such as a sensor dropping onto the floor can be filtered out or identified using approaches such as a rules-based logic model, state machine model or machine learning model. The motions after the fall can also be analyzed to characterize the severity of the fall. For example, the sensor can detect if a user continues to move on the floor, is unconscious and not moving, or is able to get back up and walk around. The location data, heart rate, galvanic skin response and other sensors can also be logged.

Once a fall is detected, and the severity of the fall analyzed (e.g., if the person is unconscious or able to get back up), the device can send an alert directly to emergency contacts, emergency response or a call center that tries to get in contact with the elderly person. Nurses, emergency contacts, adult children can also be notified via SMS, telephone call, notification, etc.

The falling characteristics such as falling speed, impact, severity, and location can be shared directly with emergency responders and emergency contacts. Impacts sustained to the body can be measured or estimated. For example, if a patient was wearing a sensor embedded device in the form of smart glasses, the glasses can measure the impact to the head during the fall. Likewise, if a sensor was located near the shoulder or pelvis, the impact can be measured and help estimate the severity of the fall.

Additionally, the fall can trigger the smart phone application, tablet, or home hub device to automatically call (via audio or video conference) the emergency contact from the user's phone. If the user has a home monitoring web camera that is connected, the system can turn on the web camera and send it GPS coordinates to help find where the user is and provide visual information to the emergency contact or emergency responders.

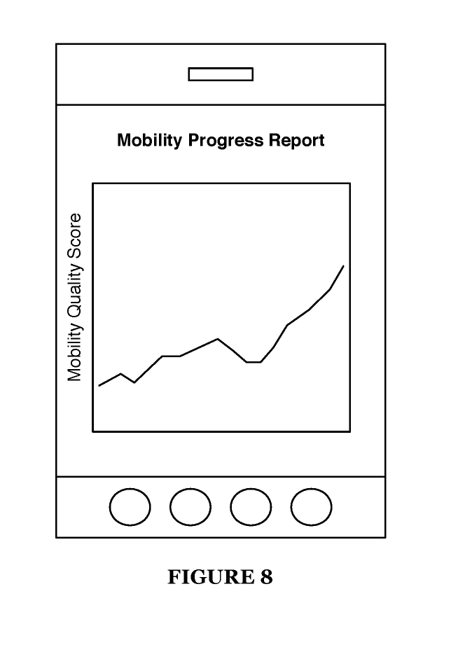

In some variations, the method can additionally be applied to enhancing mobility, which can function to coach a user for improved mobility and/or for retraining a user after the loss of mobility. In general, the method can include measuring the quality of patient mobility as reflected in the mobility metrics over an extended duration and generating a rehabilitation progress report as shown in FIG. 8. The quality of patient mobility can be a measure of positive qualities mobility metrics and the amount of desired activities (e.g., amount of walking or running). Positive qualities in mobility metrics can also be reflected by minimal fall risk assessments. The method may additionally be adapted to assisting in the rehabilitation and training of mobility in other ways.

In one variation, the method can quantify the rehabilitation progress of patients by measuring the quantity and quality of the patient's mobility metrics throughout their entire day. The system can quantify and characterize the user's biomechanical mobility quality along with syncing this data to the exact time of muscles activation activity throughout the gait cycle, giving the physiotherapist (PT) a more comprehensive understanding of the patient and their neuromuscular condition outside of the clinic. The system can be customized to detect, log or notify the PT of specific indicators important to the PT. This variation preferably includes the collection of biometric data, and more specifically the collection of muscle activity data from an EMG sensor or a suitable sensor. Biomechanical mobility quality could additionally be mapped to respiratory rate, heart rate, blood glucose levels or other sensed biometric data.

In one exemplary implementation, during patient rehabilitation, the patient needs to set up appointments for the doctor to assess their overall recovery. However, the assessments may not be enough to give a clear picture of the overall recovery as recovery can fluctuate between doctor visits. Also if the patient is doing much better, an in-person assessment may not be needed.

For example, if the user just had a hip surgery, the user may limp subtly on one side. The method will be able to quantify the asymmetry and limping nature of the patient's walk. The pelvic drop may be significantly larger on the right side, the pelvic transverse rotation may be larger than normal, and the gluteus medius muscle may not be firing correctly. The sensor can quantify the amount of time limping, the characteristics and severity of the limp, and the muscle groups that are firing or not firing throughout the gait cycle.

In another example, a physiotherapist can use the device to help accelerate a patient's process for re-learning neuro-muscular motor control (i.e., gait re-training). The device can log when the muscle groups fire at different periods of the walking gait. The data will help the physiotherapist understand which muscles are working correctly, and which ones are still inhibited. This can help locate which muscles are weak and inhibited throughout the day so that the PT can focus on treating the weakest muscle groups first.

This can accelerate the time to recover by focusing on the muscle groups that are not firing properly and adding to the biomechanical instability.

Over time, as a patient improves, the stride symmetries between the left and right side becomes more equal, the pelvis range of motion decreases, and the muscles fire at the correct times during the gait cycle. The method will be able to quantify this improvement over time, and share the data with the patient, family and physician.

While the biomechanical sensing device can track the progress of walking improvement, it can also provide real-time feedback via audio, haptic or any other communication medium to correct a patient in real-time and provide coaching and personalized tips to accelerate gait re-training outside the clinic. For example the device may come up with a personalized training plan that prioritizes the recommended adjustments that should to be made based on the importance of the metric, the progress of the user, ease of learning, or the feedback from a physician.

For example, pelvic drop may be prioritized first until the patient has mastered it before moving onto another metric to work on. In another example, patients can be reminded by the system when their pelvis becomes unstable, when their gluteus muscles are not firing correctly as indicated through the EMG sensor or when their stride becomes too large. The system can then provide another specific mechanic to work on that reduces the overall instability.

The personalized coaching can also include additional exercises or stretches to strengthen specific muscles. For instance, if the patient is walking asymmetrically on his right side and his left glutes aren't firing, the system may give the user exercises to activate and strengthen his left leg to begin balancing the stride asymmetry.

The method generated guidance can also be personalized to work with the specific physical therapist's (PT) or their gait re-training plans. The device can focus on the PT's priorities, provide customized feedback from the PT, and send the progress updates directly to the PT.

The device and system can send all this information back to the PT who can modify the training program virtually depending on the patient's progress. With this new deeper information, the PT is empowered to make decisions without having to see the patient in his clinic. The PT can then focus his time on the patients who really need to come into the clinic.

The method may additionally generate activity goals based on current mobility scores, current mobility metrics, the current fall risk assessment and/or other factors. Activity goals can be a recommended amount of walking, sitting, standing, moving, running, or other activities. For example, during a day with low risk of falling, the method may generate a recommendation of walking at least one hour that day. On a day with a moderate risk of falling, the method may update the recommendation to walk a comfortable duration four different times during the day.

The systems and methods of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

We claim:

1. A method comprising:
collecting sensor data at a biomechanical sensing device coupled to a user, wherein the sensor data includes at least accelerometer data;
performing biomechanical analysis on the sensor data and thereby generating mobility metrics of the user, wherein performing biomechanical analysis comprises:
quantifying a set of gait dynamics as a component of the mobility metrics;
processing the mobility metrics in a risk assessment model and thereby generating a fall risk assessment; and
detecting a trigger condition and triggering a response to the fall risk assessment.

2. The method of claim 1, wherein performing biomechanical analysis further comprises generating a user activity graph as a component of the mobility metrics, wherein the activity graph characterizes activity states over a time period.

3. The method of claim 1, wherein triggering a response to the fall risk assessment comprises prompting the user to rest through a feedback interface.

4. The method of claim 3, wherein generating the fall risk assessment comprises generating a rest prediction metric; and wherein prompting the user to rest further comprises prompting the user to rest for an amount of time specified by the rest prediction metric.

5. The method of claim 1, wherein detecting the trigger condition and triggering the response comprises detecting elevated risk indicated through the fall risk assessment and transmitting a communication to a caretaker.

6. The method of claim 1, wherein quantifying a set of gait dynamics comprises generating a stride shuffle metric, double-stance metric, and a tremor metric; and wherein processing the mobility metrics in a risk assessment model comprises increasing the risk of a fall in the fall risk assessment with detected increases in shuffle-associated strides, double-stance-associated strides, or the amount of tremors.

7. The method of claim 1, wherein triggering a response comprises reporting incidents from the set of: fall events, stumble events, tremor time, and double stance time.

8. The method of claim 1, further comprising measuring the quality of user mobility as reflected in the mobility metrics over an extended duration and generating a rehabilitation progress report.

9. The method of claim 1, wherein the set of gait dynamics includes gait shuffling classification; and wherein quantifying a set of gait dynamics comprises detecting vertical step displacements, classifying the gait as shuffling when vertical step displacements satisfy a shuffle condition, and thereby classifying segments of sensor data as gait shuffling.

10. The method of claim 1, wherein the set of gait dynamics includes a gait asymmetry metric; and wherein quantifying a set of gait dynamics comprises detecting right step and left step lengths, comparing the right step length and left step length, and thereby generating a gait asymmetry metric.

11. The method of claim 1, wherein the set of gait dynamics includes a gait double-stance classification; and wherein quantifying a set of gait dynamics comprises: detecting ground contact time of right steps and left steps, detecting a double-stance condition in the ground contact time of the right and left steps, and thereby generating a gait double-stance classification.

12. The method of claim 1, further comprising collecting location data of the user; and wherein the fall risk assessment is further based on the location data, wherein the risk assessment model weighs the mobility metrics differently for different location data.

13. The method of claim 1, wherein the risk assessment model weighs the mobility metrics differently at different times of day.

14. The method of claim 1, further comprising collecting temperature data; and wherein the fall risk assessment is further based on the temperature data, wherein the risk assessment model weighs the mobility metrics based in part on the temperature data.

15. The method of claim 1, wherein the risk assessment model comprises a machine learning model in classification of risk in the fall risk assessment.

16. A fall prevention system comprising:
a biomechanical sensing device that couples to a user and comprises at least an accelerometer, the sensing device being configured to collect sensor data; and
a processor configured to:
perform biomechanical analysis on the sensor data and generate mobility metrics of the user, wherein biomechanical analysis comprises configuration to: quantify a set of gait dynamics as a component of the mobility metrics, and generate a user activity graph as a component of the mobility metrics, wherein the activity graph characterizes activity states over a time period,
process the mobility metrics in a risk assessment model and generate a fall risk assessment, and
detect a trigger condition and trigger a response to the fall risk assessment.

17. The system of claim 16, further comprising a feedback interface, wherein the response to the fall risk assessment is user feedback of the current fall risk assessment that is communicated through the feedback interface.

18. The system of claim 16, wherein the user feedback is a rest recommendation.

19. The system of claim 16, wherein the gait dynamics comprises at least a stride shuffle metric, double-stance metric, and a tremor metric.

20. The system of claim 16, wherein the risk assessment model includes data inputs of location, time, and weather.

21. The system of claim 16, wherein the processor is further configured to measure the quality of patient mobility as reflected in the mobility metrics over an extended duration, and generate a rehabilitation progress report.
